(19) United States
(12) Patent Application Publication
(10) Pub. No.: US 2026/0144781 A1
(43) Pub. Date: May 28, 2026

(54) COMPOSITION AND METHOD FOR CONTROLLED HUMAN NICOTINE ABSORPTION WITHOUT PH ADJUSTERS

(71) Applicant: NIC NAC NATURALS, LLC, SPOKANE, WA (US)

(72) Inventors: NICCOLO JOHN MAGNOTTO, SPOKANE, WA (US); MARCO ANTHONY MARIA MAGNOTTO, SPOKANE, WA (US)

(73) Assignee: NIC NAC NATURALS, LLC, SPOKANE, WA (US)

(21) Appl. No.: 19/121,568

(22) PCT Filed: Oct. 23, 2023
(86) PCT No.: PCT/US2023/077560

§ 371 Date: Apr. 16, 2025

Related U.S. Application Data
(60) Provisional application No. 63/422,005, filed on Nov. 3, 2022.

(51) Int. Cl. A61K 31/465; A61K 9/20

(52) U.S. Cl. CPC A61K 31/465; A61K 9/2009; A61K 9/2013; A61K 9/2018; A61K 9/2095

(57) ABSTRACT

The invention is directed to a composition for the buccal delivery of nicotine that maintains a lower pH than is common for conventional buccal delivery systems. The composition uses a noncariogenic polyol instead of alkaline pH adjusters, and thereby avoids injury to the mouth and reduces the addiction potential. The invention also includes a method for making the composition, and a system for buccal delivery of nicotine that includes the composition.

### RELATED APPLICATIONS

This patent application is a 371 of International Application Serial No. PCT/US23/77560, filed Oct. 23, 2023, which claims the benefit of U.S. Provisional Application Ser. No. 63/422,005, filed on Nov. 3, 2022, the disclosure of which is incorporated herein by reference.

### TECHNICAL FIELD

This disclosure is directed to compositions and methods for optimizing the absorption of nicotine into the human body without the need for pH adjusters, where the nicotine is provided in a delivery system that is free of tobacco.

### BACKGROUND

Nicotine is known to have certain beneficial effects, including improved mental alertness, hunger suppression and weight control, but is addictive. Historically, the primary delivery mechanism for nicotine has been tobacco, including cigarettes, cigars, pipes and chewing tobacco. The addictive nature of nicotine often leads to prolonged, habitual use of tobacco products, resulting in cancer and other health ailments. However, it has been discovered that the adverse health effects of tobacco are caused primarily by other ingredients in the tobacco.

In order to enable the beneficial use of nicotine without the adverse health effects of tobacco, alternative delivery methods for nicotine have been developed for oral use. Such methods have relied on the use of pH adjusters, for example sodium carbonate, which increase the pH in the user's mouth to enhance the oral absorption of nicotine. Common nicotine pouches can result in highly alkaline saliva pH's adjacent to the tender skin tissues on the mouth. For example, the alkaline pH adjusters contact the tender skin in the gum, buccal and sublingual regions and can have undesirable side effects. For example, pH adjusters can agitate the gums and buccal tissues and cause gingival degradation. Furthermore, inflammation due to alkaline burn may accelerate the nicotine delivery to an unintended high rate, raising the addiction potential. Excessive alkalinity can also result in gingival burns, which accelerate the nicotine delivery and increase the addiction potential. The overall negative impacts excessive alkalinity, increased addiction potential, and damage to long-term oral health have limited or discouraged the use of these alternative nicotine delivery systems.

The delivery mechanism of pH adjusters such as sodium carbonate actually relies on high alkalinity creating minor oral injury to enhance the absorption of nicotine. Sodium carbonate is used to mildly burn and ultimately inflame the buccal tissue drawing blood to the area and in turn increasing nicotine delivery to the brain. This “rush” of rapidly absorbed nicotine is directly associated with increased addiction potential.

In view of the foregoing, there is a need or desire for an oral nicotine delivery system that reduces or eliminates injury to the user's mouth.

There is also a need or desire for an oral nicotine delivery system that moderates the nicotine delivery rate and, hence, the addiction potential.

### SUMMARY

The invention is directed to a composition for the buccal delivery of nicotine that maintains a controlled saliva pH, especially in the sensitive contact areas inside the user's mouth. The composition provides for effective nicotine delivery while eliminating the need for alkaline pH adjusters, and avoids burns to the skin inside the mouth. The composition thereby avoids injury to the mouth and reduces the addiction potential. The invention also includes a method for making the composition, and a system for buccal delivery of nicotine that includes the composition.

The invention embodies the discovery that alkaline pH adjusters can be eliminated from the composition and can be replaced with a non-cariogenic polyol that provides a lower pH in the range of about 7.0 to about 10.0. The lower pH significantly reduces or avoids the inflammation and other injuries to the gums and buccal tissues and reduced the addiction potential of the nicotine. Non-cariogenic polyols do not contribute to any acid formation and do not produce caries, or tooth decay, because they are not metabolized by microorganisms in plaque. Non-cariogenic polyols have surprisingly been found to serve as effective delivery vehicles for nicotine at the neutral or slightly alkaline pH.

The composition can be provided in the form of a tablet, lozenge, or a powder encapsulated in a water-soluble pouch, that serves as a delivery vehicle. The delivery vehicle can be inserted in the buccal regions of the mouth, which include the region between the cheeks, lips and gums of the human mouth, and the sublingual region. The composition can be devoid of alkaline pH adjusters, which specifically encompass the carbonate, bicarbonate, oxide and hydroxide salts of sodium, potassium, calcium, and magnesium.

With the foregoing in mind, it is a feature and advantage to provide a composition for the buccal delivery of nicotine that includes:
about 0.1% to about 50% by weight of a nicotine compound; and
about 50% to 99.9% by weight of a non-cariogenic polyol;
wherein the composition is devoid of alkaline pH adjusters.

It is also a feature and advantage to provide a method of making a composition for the buccal delivery of nicotine, that includes the following steps:
providing a nicotine compound selected from the group consisting of nicotine, nicotine polacrilex, nicotine bitartrate dehydrate, salts of the foregoing, and combinations thereof;
providing a non-cariogenic polyol selected from the group consisting of xylitol, erythritol, D-arabitol, sorbitol, and combinations thereof; and
mixing the nicotine compound and non-cariogenic polyol together to form the composition.

It is also a feature and advantage to provide a delivery system for administering a composition for the buccal delivery of nicotine, that includes:
the composition; and
a delivery mechanism;
wherein the composition comprises about 0.1% to about 50% by weight of a nicotine compound and about 50% to 99.9% by weight of a non-cariogenic polyol and is devoid of alkaline pH adjusters; and
the delivery mechanism is a tablet, a lozenge, or a pouch.

The foregoing and other features and advantages will become further apparent from the following Detailed Description.

### DETAILED DESCRIPTION

In one embodiment, the invention is directed to a composition for the buccal delivery of nicotine. The composition includes about 0.1% to about 50% by weight of a nicotine compound and about 50% to about 99.9% by weight of a non-cariogenic polyol and can be devoid of alkaline pH adjusters.

The nicotine compound can be in the form of pure nicotine, nicotine polacrilex, nicotine bitartrate dehydrate, salts of the foregoing, and combinations thereof. Nicotine salts can be extracted from tobacco leaves and bonded with benzoic acid. Examples of nicotine salts include nicotine lactate, nicotine salicylate, nicotine benzoate, and nicotine levulinate. The nicotine compound can be present in the composition in an amount of at least about 0.1% by weight, or at least about 0.3% by weight, or at least about 0.5% by weight, or at least about 1% by weight, or at least about 5% by weight, or at least about 10% by weight; and/or up to about 50% by weight, or up to about 40% by weight, or up to about 30% by weight, or up to about 25% by weight, or up to about 20% by weight, or up to about 15% by weight.

The non-cariogenic polyol can be in the form of xylitol, erythritol, D-arabitol, sorbitol, or a combination of the foregoing. The non-cariogenic polyol can suitably include xylitol. The non-cariogenic polyol can be present in the composition in an amount of at least about 50% by weight, or at least about 60% by weight, or at least about 70% by weight, or at least about 75% by weight, or at least about 80% by weight, or at least about 85% by weight; and/or up to about 99.9% by weight, or up to about 99.7% by weight, or up to about 99.5% by weight, or up to about 99% by weight, or up to about 95% by weight, or up to about 90% by weight.

The types and amounts of the nicotine compound and non-cariogenic polyol can be selected as needed to provide the buccal delivery composition which, upon dissolution in the user's mouth, results in a saliva pH of about 7.0 to about 10.0, or about 8.0 to about 10.0, or about 8.5 to about 9.5, especially in the sensitive gum, buccal and/or sublingual areas of the user's mouth that are adjacent to the composition. One objective is to maintain the pH at a controlled lower level than the highly alkaline pHs that resulted from common prior art buccal delivery compositions. The buccal delivery composition can be devoid of the alkaline pH adjusters that were conventionally used to achieve the high pH's. For example, the buccal delivery composition can be devoid of alkaline pH adjusters such as the carbonate, bicarbonate, oxide, and hydroxide salts of sodium, potassium, calcium, and magnesium.

The nicotine compound and the non-cariogenic polyol can be blended together, dried, and processed to provide the composition in the form of tablets, lozenges, or powder which can then be encapsulated in water-soluble pouches. Additional ingredients can be added as needed to adapt the composition to its intended form. For example, flavor ingredients can be added to provide the taste of mint or another confectionary item. Fillers and bonding agents can be added, as needed, to enable the formation and pressing of tablets or lozenges. Tablets, lozenges, and pouches can have suitable sizes and thicknesses to enable comfortable placement in the user's mouth. For example, a tablet or lozenge can have a thickness of at least about 1 mm, or at least about 3 mm, and/or less than about 7 mm, or less than about 5 mm, to enable comfortable placement adjacent to the upper and/or lower gums of humans. This will enable sufficient buccal absorption of nicotine y the user without the aid of alkaline pH adjusters.

Suitable additional ingredients can include fillers, mild organic acids, and flavor ingredients that provide enhanced physical stability and flavor to the composition. For example, the composition can include about 1% to about 7% by weight stearic acid, suitable about 2% to about 5% by weight. The composition can include silica or another filler in an amount needed to absorb and powderize the nicotine and flavor (if present). In one embodiment, silica can be present in an amount of about 0.1% to about 1% by weight, suitably about 0.2% to about 0.5% by weight. Higher amounts of silica, sufficient to absorb the nicotine, can be used if the nicotine is used in relatively high amounts. In one embodiment, the amount of silica can be about 30% to 60%, suitably about 40% to 50%, of the amount of nicotine in the composition. For example, if the nicotine constitutes 20% by weight of the composition, then the silica can constitute about 6% to about 12%, or about 8% to about 10% of the composition. The composition can include about 0.1% to about 1% by weight of a flavor ingredient, such as an essential oil, suitably about 0.1% to about 0.5% by weight.

Particularly suitable embodiments of the composition can contain xylitol as the non-cariogenic polyol. In one embodiment, the xylitol can be present in about 93% to about 98% by weight, sufficient to render the composition compressible. This composition can include about 0.35 to about 0.9% by weight nicotine, about 2% to about 5% by weight stearic acid, about 0.1% to about 0.5% by weight silica, and zero or 0.1% by weight to about 0.4% by weight of an essential oil flavor ingredient.

In order to produce the composition for the buccal delivery of nicotine, the silica can be first combined with the nicotine and flavor (if any) in an amount sufficient to absorb and powderize the nicotine and flavor, which are otherwise liquid. The resulting powder can be lightly milled and broken up using an oscillating granulator. The powder can then be blended using a cone blender equipped with an intensifier bar. The powder can then be placed in water-soluble delivery pouches or, alternatively, can be run through a rotary tablet press to form tablets and lozenges.

During use, the tablet, lozenge, or water-soluble pouch can be placed in the buccal region of the mouth adjacent to the gums, or in the sublingual region under the tongue, and allowed to dissolve without agitation. As the composition dissolves, the nicotine is released and absorbs through the adjacent tissue. The non-cariogenic polyol facilitates the absorption through the adjacent tissue by reducing oral acidity without the use of alkaline pH adjusters.

### EXAMPLE

Compositions containing 712.5-720 mg xylitol, 3 mg or 6 mg of pure nicotine, 15 to 37 mg of stearic acid, 0.75 to 3.75 grams of silica and 0.75 to 3 grams of flavor were mixed using a pharmaceutical blender and formed into tablets weighing approximately750 mg using a rotary tablet press. The tablets contained approximately 0.4% and 0.8% by weight nicotine, respectively. The tablets were administered to five subjects by placing them in the buccal region adjacent to the gums. Saliva samples were tested after dissolution of each tablet using pH test strips placed between the gum and buccal tissues of the user's mouths. The saliva pH's averaged 9.2 and ranged from 8 to 10, and the users experienced no discomfort or injury in the gum or buccal regions.

The embodiments of the invention described herein are exemplary. Various modifications and improvements can be made without departing from the spirit and scope of the invention. The scope of the invention is defined by the appended claims, and all changes that fall within the meaning and range of equivalents ae intended to be embraced therein.

1. A composition for the buccal delivery of nicotine, comprising:
about 0.1% to about 50% by weight of a nicotine compound; and
about 50% to 99.9% by weight of a non-cariogenic polyol;
wherein the composition is devoid of alkaline pH adjusters.

2. The composition of claim 1, wherein the nicotine compound is selected from the group consisting of nicotine, nicotine polacrilex, nicotine bitartrate dehydrate, nicotine salts, and combinations thereof.

3. The composition of claim 1, wherein the non-cariogenic polyol is selected from the group consisting of xylitol, erythritol. D-arabitol, sorbitol, and combinations thereof.

4. The composition of claim 1, wherein the non-cariogenic polyol comprises xylitol.

5. The composition of claim 1, comprising:
about 5% to about 40% by weight of a nicotine compound; and
about 60% to 95% by weight of a non-cariogenic polyol.

6. The composition of claim 1, comprising:
about 10% to about 30% by weight of a nicotine compound; and
about 70% to 90% by weight of a non-cariogenic polyol.

7. The composition of claim 1, wherein the composition yields a saliva pH of about 7.0 to about 10.0.

8. The composition of claim 1, wherein the composition yields a saliva pH of about 8.0 to about 10.0.

9. The composition of claim 1, wherein the composition is formed as a tablet.

10. The composition of claim 1, wherein the composition is formed as a lozenge.

11. The composition of claim 1, wherein the composition is in the form of a powder enclosed in a pouch.

12. A method for making a composition for the buccal delivery of nicotine, comprising the steps of:
providing a nicotine compound selected from the group consisting of nicotine, nicotine polacrilex, nicotine bitartrate dehydrate, salts of the foregoing, and combinations thereof;
providing a non-cariogenic polyol selected from the group consisting of xylitol, erythritol, D-arabitol, sorbitol, and combinations thereof; and
mixing the nicotine compound and non-cariogenic polyol together to form the composition.

13. The method of claim 12, further comprising the step of forming the composition into a tablet.

14. The method of claim 12, further comprising the step of forming the composition into a lozenge.

15. The method of claim 12, further comprising the steps of forming the composition into a powder and placing the powder in a water-soluble pouch.

16. The method of claim 12, wherein the composition yields a saliva pH a pH of about 7.0 to about 10.0.

17. The method of claim 12, wherein the composition yields a saliva pH of about 8.0 to about 10.0.

18. A delivery system for administering a composition for the buccal delivery of nicotine, comprising:
the composition; and
a delivery mechanism;
wherein the composition comprises about 0.1% to about 50% by weight of a nicotine compound and about 50% to 99.9% by weight of a non-cariogenic polyol and is devoid of alkaline pH adjusters; and
the delivery mechanism is a tablet, a lozenge, or a pouch.

19. The delivery system of claim 18, wherein the nicotine compound is selected from the group consisting of nicotine, nicotine polacrilex, nicotine bitartrate dehydrate, nicotine salts, and combinations thereof.

20. The delivery system of claim 18, wherein the non-cariogenic polyol is selected from the group consisting of xylitol, erythritol. D-arabitol, sorbitol, and combinations thereof.

21. The delivery system of claim 18, wherein the composition has a pH of about 7.0 to about 10.0.
